(19) United States
(12) Patent Application Publication
(10) Pub. No.: US 2005/0249760 A1
(43) Pub. Date: Nov. 10, 2005

(54) Human body affinitive lubricating protective and repair gel

(76) Inventors: Sun Shin-Ching, Taipei (TW); Chiang Chao-Wang, Taipei (TW)

Correspondence Address: Sun Shin-Ching, P. O. Box 24-108, Taipei, TW

(21) Appl. No.: 10/837,585

(22) Filed: May 4, 2004

(51) Int. Cl. A61K 33/42; A61K 33/38

(52) U.S. Cl. 424/401; 424/618

(57) ABSTRACT

The present invention discloses a human body affinitive lubricating protective and repair gel, which combines photocatalyst (TiO₂) particles with a disinfection material such as nano silver to produce a TiO₂Ag compound, and mixes the compound with a filtered natural biological organism with a molecular weight below 50 Kbp and a concentration of 0.05˜5% including small molecules of collagen, chitin, chitosan, hyaluronic acid, vitamin C, and nano iron, and its pH value is adjusted to 6 which is close to the pH value of our skin, and thus the mixture constitutes a gel affinitive to human skins and non-toxic to human cells. An accessory ingredient such as an aromatic agent, a detergent, or a cosmetic agent is added in the manufacturing process to produce an emulsion or a paste for coating or spraying, which is used as a health or sanitation item such as a hand liquid soap, a cosmetic, a shampoo, or a disinfectant. The feature of the small diameter of the particles (1˜100 n/m) can penetrate the cuticle of our skin and remain at the derma inside the skin pores to produce a protective film, and the collagen can be absorbed by the skin to give the effects of repairing tissues, stopping bleeding, and lubricating skins. With the cyclic disinfection effect of the TiO₂Ag compound, a deeper clean skin to prevent germs is obtained and a disinfection effect is achieved to eliminate acnes and pimples. The present invention also makes use of an oxidation-reduction mechanism of the photocatalyst (TiO2) to achieve long-hour disinfection and naturally remove germs.

### BACKGROUND OF THE INVENTION

I. Field of the Invention

The present invention relates to lubricating gels, more particularly to a human body affinitive lubricating protective and repair gel for cleaning human bodies and maintaining and protecting our health.

The present invention combines photocatalyst (TiO₂) particles with a disinfection material such as nano silver to produce a nano TiO₂Ag compound, and mixes the compound with a filtered natural biological organism with a molecular weight below 50 Kbp and a concentration of 0.05˜5% including small molecules of collagens, chitin, chitosan, hyaluronic acid, vitamin C, and nano iron, and its pH value is adjusted to 6 which is close to the pH value of our skin, and thus the mixture constitutes a gel affinitive to human skins and non-toxic to human cells. An accessory ingredient such as an aromatic agent, a detergent, or a cosmetic agent is added in the manufacturing process to produce an emulsion or a paste for coating or spraying, which is used as a health or sanitation item such as a hand liquid soap, a cosmetic, a shampoo, or a disinfectant. The feature of the small diameter of the particles (1˜100 n/m) can penetrate the cuticle of our skin and remain at the derma inside the skin pores to produce a protective film, and the collagen can be absorbed by the skin to give the effects of repairing tissues, stopping bleeding, and lubricating skins. With the cyclic disinfection effect of the TiO₂Ag compound to produce a deeper clean skin to prevent germs and have the disinfection effect to eliminate acnes and pimples, and also makes use of the oxidation-reduction mechanism of the photocatalyst TiO2 to achieve the long-hour disinfection and naturally remove germs.

II. Description of the Prior Art

Heretofore, it is known that most of the present lubricants (such as the lubricant for condoms) adopt inorganic silicon oils which will lose the lubricating effect very shortly after its use due to its hydrophobic and biological non-affinitive properties. Since the lubricant used for condoms primarily intends to slow down the activity of sperms, it does not pay much attention on preventing the infections by viruses, germs, and fungi, and does not take biological affinity and decomposability into consideration, and thus creating an incomplete and inapplicable issue at a later stage. Since the emulsion used for cleaning and sanitation (such as hand soap, hand lotion, cosmetic, shampoo, or disinfection liquid) is an item often used by people and its molecular size is too big, therefore such emulsion is usually applied to the external surface of our skin only. The emulsion will be contaminated easily; the effect cannot be lasted long; and it will cause uncomfortable feeling to users. The effects on repairing tissues, stopping bleeding, and lubricating skins are very limited. There are many kinds of germs, and it is not easy to kill the infective viruses such as SARS and herpes. Traditionally, the viruses are removed by the interference, deactivation, or anti-growth method, and thus deriving many side effects such as drug resistance, mutation, and strengthening toxic viruses. Even some germs or viruses can be killed, but there are problems at dead corners or complete cleaning generally causing germs, viruses, or their dead bodies to be remained on the skin. Germs and viruses will be bred easily when cleaning is not done properly, and will be accumulated days after days to cause infections, stinks, and decays. Such problem definitely needs to be studied and improved.

### SUMMARY OF THE INVENTION

In view of the foregoing shortcomings, the inventor of the present invention based on years of experience on the cleaning, health, and cosmetic products to conduct extensive researches and experiments and find a way to overcome the shortcomings, and finally invented the a human body affinitive lubricating protective and repair gel in accordance with the present invention.

It is therefore a primary object of the invention to provide a human body affinitive lubricating protective and repair gel, which combines photocatalyst (TiO₂) particles with a disinfection material such as nano silver to produce a TiO₂Ag compound, and mixes the compound with a filtered natural biological organism with a molecular weight below 50 Kbp and a concentration of 0.05˜5% including small molecules of collagens, chitin, chitosan, hyaluronic acid, vitamin C, and nano iron, and its pH value is adjusted to 6 which is close to the pH value of our skin, and thus the mixture constitutes a gel affinitive to human skins and non-toxic to human cells. An accessory ingredient such as an aromatic agent, a detergent, or a cosmetic agent is added in the manufacturing process to produce an emulsion or a paste for coating or spraying, which is used as a health or sanitation item such as a hand liquid soap, a cosmetic, a shampoo, or a disinfectant. The feature of the small diameter of the particles (1˜100 n/m) can penetrate the cuticle of our skin and remain at the derma inside the skin pores to produce a protective film, and the collagen, vitamin C, and nano iron can be absorbed by the skin to provide the effects of repairing tissues, stopping bleeding, and lubricating skins.

It is still another object of the invention to provide a human body affinitive lubricating protective and repair gel that contains a TiO₂Ag compound. With the cyclic disinfection effect of the TiO₂Ag compound, a deeper clean skin is obtained to prevent germs and the disinfection effect is achieved to eliminate acnes and pimples, and the present invention also makes use of the oxidation-reduction mechanism of the photocatalyst TiO2 to achieve a long-hour disinfection by reduction and decompose germs and viruses into water and carbon dioxide by oxidation, and also naturally remove germs to avoid the problems of infections, stinks, and decays.

### BRIEF DESCRIPTION OF THE DRAWING

FIG. 1 is a flow chart of the present invention;

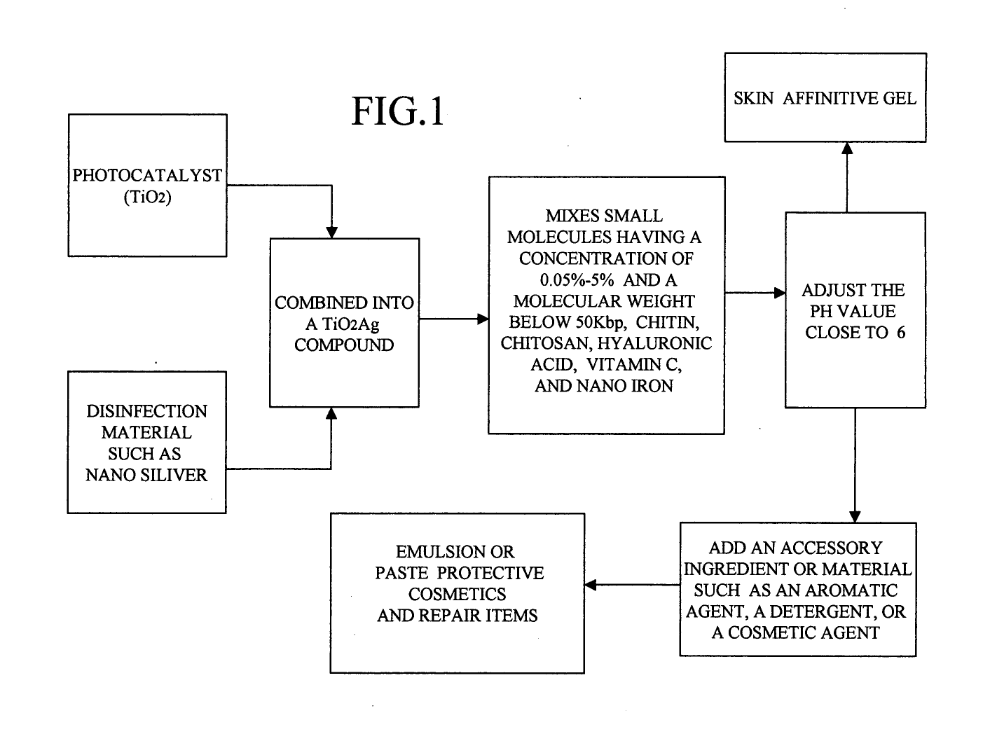

### DESCRIPTION OF THE PREFERRED EMBODIMENT

The accomplishment of the above-mentioned object of the present invention will become apparent from the following description and its accompanying drawings which disclose illustrative an embodiment of the present invention, and are as follows:

The present invention combines photocatalyst (TiO₂) particles with a disinfection material such as nano silver to produce a TiO₂Ag compound, and mixes the compound with a filtered natural biological organism with a molecular weight below 50 Kbp and a concentration of 0.05˜5% including small molecules of collagens, chitin, chitosan, hyaluronic acid, vitamin C, and nano iron, and its pH value is adjusted to 6 which is close to the pH value of our skin, and thus the mixture constitutes a gel affinitive to human skins and non-toxic to human cells to serve as a lubricating protective lotion. An accessory ingredient such as an aromatic agent, a detergent, or a cosmetic agent is added in the manufacturing process to produce an emulsion or a paste for coating or spraying, which is used as a hand liquid soap, a cosmetic, a shampoo, a disinfectant, or a health/sanitation item

Since the size of the disinfection materials including the photocatalyst (TiO₂) and the nano silver below 50 n/m are combined to produce the TiO₂Ag with a diameter below 1100 n/m and the size of the viruses is about 100˜200 n/m and the gap of the skin pores is about 200 n/m, therefore after the gel is coated or sprayed, the gel will penetrate into the cuticle of our skin and remain at the derma inside the skin pores to produce a protective film with a large surface area and a better activation. If a virus or an AID virus penetrates the skin pores or a wound, the strong oxidability of the photocatalyst (TiO₂) and the disinfection effect of the disinfectant such as the silver ion can kill hundreds of different germs and viruses. Please refer to Attachment 1 for the test report on such effect. Further, the photocatalyst (TiO₂) has the strong oxidation-reduction feature and is capable of decomposing organic matters, germs, and viruses into water and carbon dioxide by oxidation to naturally remove germs and viruses and avoid the germs and viruses from remaining on the skin, stinks and mutation of germs. After absorbing infrared light, the photocatalyst (TiO₂) can be reduced and used repeatedly. Therefore, the photocatalyst can reduce the harm of infrared light to human bodies, and the collagen, vitamin C, and nano iron can be absorbed by the skin to give the effects of producing long-hour biological lubrication effect (similar to the lubrication) and repairing biological tissues, stopping bleeding, and curing wounds. Since the gel comes from a natural living organism, the adverse effects for being allergic and unsuitable for the nature can be reduced. Besides being used as the biological affinitive lubricant for condoms, the gel can be used as an invisible condom and gloves.

If hand soap, shampoo or liquid soap is added into the gel according to the invention to act as a denaturing agent, the moisturizing and lubricating effects for human bodies and hair can be improved. The collagen and chitin give decoration and static charge balancing effects on hair, release pain from damaged skin, stop bleeding, and repair and cure wounds quickly. The TiO₂Ag can remove the germs in skin pores and scalp, and thus can release itching for a long time and disinfect at a deep layer. The reaction of the TiO₂Ag compound is activated to remove the chlorine remained on the scalp or hair first in order to eliminate the damage caused by the remaining chlorine. The hand soap or the liquid soap after being rinsed form an invisible protective film on our body or hands to prevent them from being intruded by germs and viruses. In the meantime, the soap also supplies moisture to dry hands and body surface caused by the rinses and prevents the skin from being pulled tightly to provide the repairing, moisturizing, and adjusting functions by the collagen and chitin compound of a low molecular weight. The coupling agent of present hair dyes and the dye intermediate oxidant usually use active chemicals such as a strong alkaline ammonia solution, hydrogen peroxide solution, or metal salts, but these chemicals usually cause damages and inflammation to hair cuticle, fiber, and skin. Experiments show that the TiO₂Ag compound uses its gentle long-hour oxidation-reduction mechanism to substitute the hydrogen peroxide or decrease its consumption and thus reducing the harm to human bodies. The chitin can effectively absorb remained heavy metals and harmful matters, and the collagen having a low molecular weight can maintain and repair damaged skin, and thus minimizing the damage caused by hair dyes.

In summation of the description above, the present invention discloses a novel method to provide a human body affinitive protective and repair gel for minimizing the damage as well as giving the effects of repairing, maintaining, and protecting deep layers. The present invention complies with the patentability, and thus is submitted to the Patent and Trademark Office for review and granting of the commensurate patent rights.

While a preferred embodiment of the invention has been shown and described in detail, it will be readily understood and appreciated that numerous omissions, changes and additions may be made without departing from the spirit and scope of the invention.

1. A human body affinitive lubricating protective and repair gel, combining a photocatalyst (TiO₂) particle with a disinfection material such as nano silver to produce a nano TiO₂Ag compound, and mixing said nano TiO₂Ag compound with a filtered natural biological organism having a molecular weight below 50 Kbp and a concentration of 0.05˜5% and being comprised of small molecules of collagen, chitin, chitosan, hyaluronic acid, vitamin C, and nano iron, and its pH value being adjusted to 6 which is close to the pH value of our skin, thereby constituting a gel affinitive to human skins and non-toxic to human cells.

2. The human body affinitive lubricating protective and repair gel of claim 1, further comprising an accessory ingredient selected from a collection of an aromatic agent, a detergent, and a cosmetic agent to selectively form an emulsion and a paste to serve as a health item selected from a collection of a hand liquid soap, a cosmetic, a shampoo and a disinfectant.
